(19) United States
(12) Patent Application Publication
(10) Pub. No.: US 2011/0160632 A1
(43) Pub. Date: Jun. 30, 2011

(54) ANKLE SPLINT

(76) Inventors: Thomas Beckers, Chaponost (FR); Gerard Anglada, Saint Etienne (FR)

(21) Appl. No.: 12/991,030

(22) PCT Filed: Mar. 20, 2009
(86) PCT No.: PCT/FR2009/050473

§ 371 Date: Feb. 22, 2011

(30) Foreign Application Priority Data
May 5, 2008 (FR) 08/02474

(51) Int. Cl. A61F 5/052

(52) U.S. Cl. 602/27

(57) ABSTRACT

The splint comprises, on the one hand, a shell intended to be positioned against the external face of the leg and, on the other hand, an L-shaped support comprising an upright intended to be positioned against the external face of the foot and a tongue intended to be positioned under the foot. Furthermore, the splint comprises a connecting piece connecting the shell and the support, mounted so that it can pivot with respect to the shell about a first transverse axis of articulation and mounted such that it can pivot with respect to the support about a second transverse axis of articulation, the first and second axes of articulation being offset from one another vertically and in the anterioposterior direction, in the neutral position of use.

The present invention relates to an ankle splint, more particularly intended for treating a sprain of the external lateral ligament of the ankle.

Splints used for this injury are already known.

On the one hand, the document EP 1 234 560 proposes an ankle splint comprising an L-shaped shell having an upright bearing against the leg and a transverse tongue positioned under the heel. However, since the shell is monolithic, this splint serves essentially to immobilise the ankle joint and is not entirely satisfactory in terms of allowing a user to walk.

On the other hand, some splints are designed with an articulation between the part positioned against the leg and the part positioned against the foot. This type of splint therefore allows a gradual return to activity for the user, while continuing to ensure a certain hold that prevents the reoccurrence of the injury. However, this splint provided with a single axis of articulation has in particular the following disadvantages:

the impact of the foot on the ground, when walking, is transmitted into the leg without any damping, which is a source of discomfort and possibly aggravation of the injury;

in order to properly accompany the movements of the ankle and thus to obtain a good level of comfort for the user, it would be necessary to make the axis of articulation of the ankle in flexion/extension coincide with the axis of articulation of the splint. However, this is very difficult or even impossible to achieve in practice.

Furthermore, the document U.S. Pat. No. 2,477,591 proposes a splint that is intended to raise the foot of the user so as to prevent it from hitting the ground. This splint comprises a part positioned against the leg and a part positioned against the foot, these parts being articulated with respect to one another by way of two pivot axes.

The object of the invention is to overcome the disadvantages mentioned above by providing an ankle splint that aims to prevent varus, which allows a user to return to physical activity and provides said user with a high degree of comfort during use.

To this end, the invention relates to an ankle splint comprising:

a shell intended to be positioned against the external face of the leg;

an L-shaped support comprising an upright intended to be positioned against the external face of the foot and a tongue intended to be positioned under the foot;

a connecting piece connecting the shell and the support, said connecting piece being mounted such that it can pivot with respect to the shell about a first transverse axis of articulation and mounted such that it can pivot with respect to the support about a second transverse axis of articulation.

According to a general definition of the invention, this splint is such that the first and second axes of articulation are offset from one another vertically and in the anteroposterior direction when the splint is positioned on the ankle of a user, said ankle being in the neutral position.

The neutral position of use of the splint is defined as being the position in which the splint is attached to a user who is in the standing position (hence with the leg substantially vertical), and who has the ankle in the neutral position, that is to say neither in flexion nor in extension. In this position, the transverse direction is defined as being the internal-external direction of the user.

In practice, the connecting piece is located opposite the lateral malleolus and the two axes of articulation are located close to the axis of articulation of the ankle.

The presence of two offset axes of articulation on the splint makes it possible on the one hand to generate an effect of damping the impact of the heel on the ground when walking, and on the other hand to accompany much more effectively the flexion/extension movements of the ankle, by liberating the articulation of the ankle. Thus, the splint according to the invention has a much improved wearing comfort compared to the prior art. In addition, it allows a user to return to a sporting activity while benefiting from a hold to his ankle that prevents any reoccurrence of the injury.

Preferably, the second axis of articulation is offset towards the front relative to the first axis of articulation.

In the neutral position of use, the shell and the upright of the support may extend in substantially vertical directions which are offset from one another in the anteroposterior direction.

Advantageously, the splint furthermore comprises a bearing member arranged opposite the internal face of the connecting piece and attached to the shell and to the support, said bearing member, made from a semi-rigid material, being designed to keep the shell and the support in the neutral position of use when the ankle is in the neutral position, to be able to be elastically deformed so as to allow the pivoting of the connecting piece about the first and second axes of articulation when the ankle is in flexion or in extension and, from this deformed position, to return the splint to its neutral position when the ankle returns to the neutral position.

The bearing member may have a concave internal face which substantially mates with the shape of the lateral malleolus. This shape, combined with the fact that the material forming the bearing member is semi-rigid, contributes to the wearing comfort of the splint for the user.

The splint may furthermore comprise a heel cushion intended to be assembled to the tongue of the support and to be positioned under the foot.

According to one possible embodiment, the heel cushion comprises a sole intended to be positioned under the foot, which sole has a transverse cavity that is open in the downward direction in the position of use, said cavity being designed to receive the tongue of the support so that, in the mounted position, the lower face of the tongue of the support is located substantially in the same plane as the lower face of the sole. The foot positioned in the splint thus rests on a flat lower face without any raised areas.

Advantageously, the sole and the tongue of the support may comprise means for reciprocal assembly by engagement, such as: at least one stud on one part engaging in a hole formed in the other part; at least one boss extending in a horizontal plane (in the position of use) on one part and being housed in a corresponding recessed portion of the other part; etc.

The heel cushion comprises for example an internal lateral flange and/or external lateral flange and/or a posterior flange, which makes it possible to limit or completely prevent the displacement of the foot relative to the heel cushion. In the case where the three flanges—internal lateral, external lateral and posterior—are present, the heel cushion forms a housing for the foot, and in particular for the heel, which is therefore well-positioned and held. By providing a heel cushion that has a housing for receiving the heel, the invention ensures a very good stabilisation of the ankle, which makes it possible for the splint to be equipped with just one shell (on just one side of the leg).

Nevertheless, the splint may comprise an additional shell intended to be positioned against the internal face of the leg, substantially opposite the shell, by being assembled to this shell by at least one strap.

One possible embodiment of the invention will now be described, by way of non-limiting example, with reference to the appended figures:

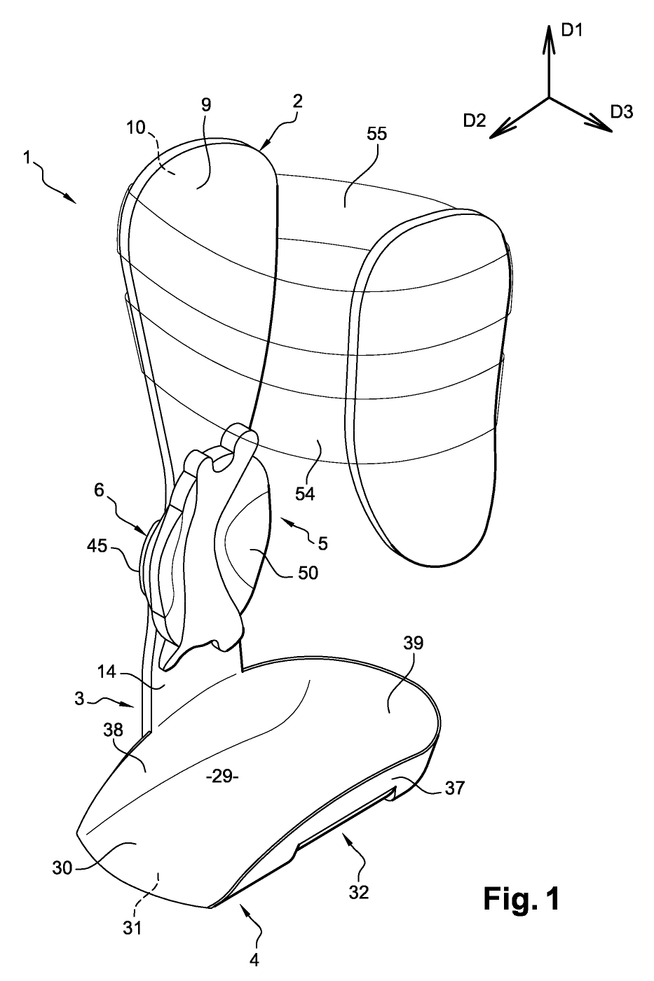

FIG. 1 is a perspective view of a splint according to the invention;

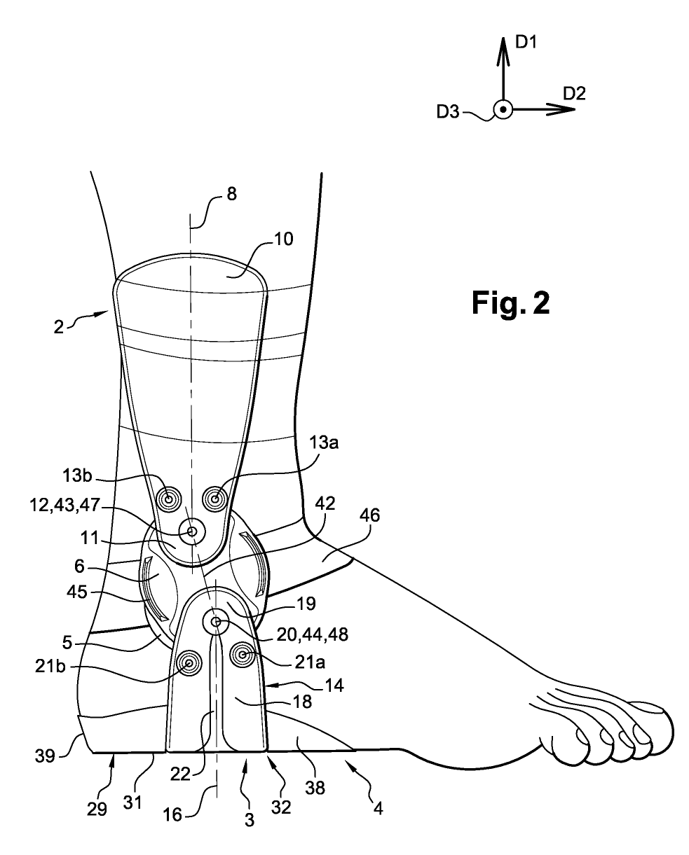

FIG. 2 is an external side view of this splint, positioned on the ankle of a user;

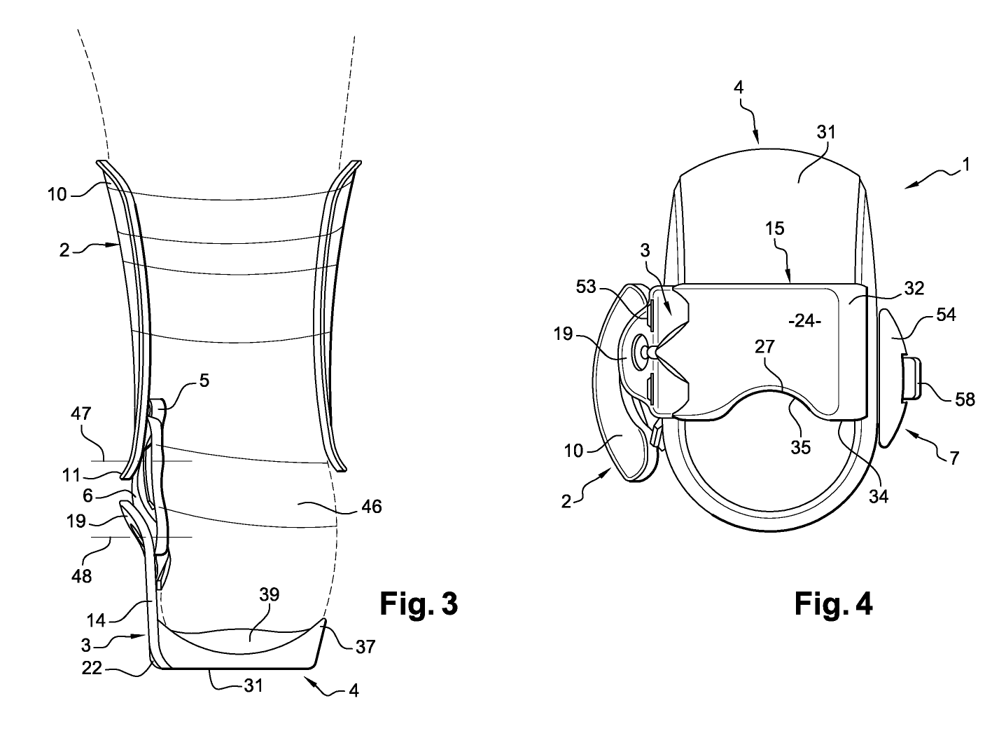

FIG. 3 is a front view of this splint, positioned on the ankle of a user;

FIG. 4 is a view of this splint from below;

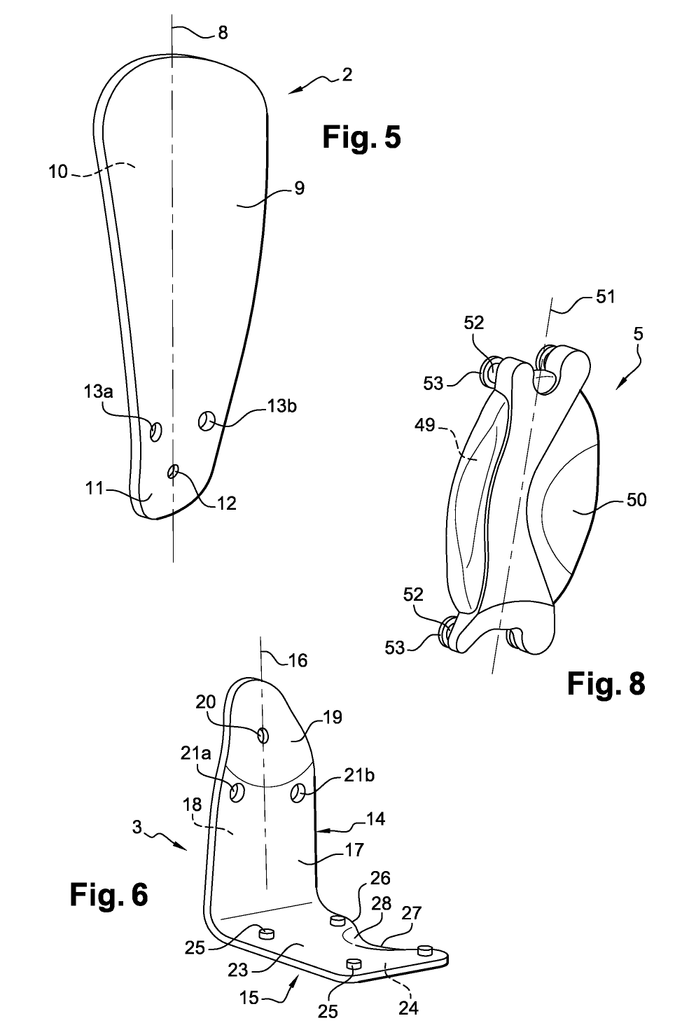

FIGS. 5 to 9 are perspective views of different constituent elements of the splint, namely respectively: the shell, the support, the heel cushion, the external bearing member, the connecting piece and the additional (internal) shell.

FIG. 1 shows a splint 1 according to the invention which comprises an external shell 2, a support 3, a heel cushion 4, an external bearing member 5, a connecting piece 6 and an internal shell 7.

The splint 1 will be described in the neutral position of use, as shown in FIGS. 1 to 4. The vertical direction D1, the anteroposterior direction D2 and the transverse direction D3 (internal-external) are defined.

The shell 2, more particularly shown in FIG. 5, is a part made from rigid material, for example made from polyamide, which is intended to be positioned against the external face of the leg. The shell 2 has a vertically elongated shape, the width of which (along D2) increases from the bottom upwards. The shell 2 has a vertical axis of symmetry 8, an internal face 9 and an external face 10. The internal face 9 has a “horse's saddle” type shape, that is to say having both a shape curved about a vertical direction, in order to fit the generally cylindrical shape of the leg, and a shape that is curved in the lower part about an anteroposterior direction, creating a lower end part 11 which is bent outwards and which will be positioned on the lateral malleolus.

The shell 2 is furthermore provided with three through-orifices: one orifice 12 located on the axis of symmetry 8, in the lower end part 11, and two orifices 13a, 13b located above the orifice 12, on each side of the axis of symmetry 8 and substantially aligned along D2.

The support 3, more particularly shown in FIG. 6, is a part made from rigid material, for example made from polyamide, which has an L-shape comprising an upright 14 intended to be positioned against the external face of the foot and a tongue 15 intended to be positioned under the foot. The upright 14 and the tongue 15 are substantially perpendicular and connected by a fillet.

The upright 14 has a vertical axis of symmetry 16, an internal face 17 and an external face 18. The upper end part 19 of the upright 14 is bent outwards and has a shape that is curved about a vertical direction so as to be able to be positioned on the lateral malleolus. The upright 14 is furthermore provided with three through-orifices: one orifice 20 located on the axis of symmetry 16, in the upper end part 19, and two orifices 21a, 21b located below the orifice 20, on each side of the axis of symmetry 16, the anterior orifice 21a being higher than the posterior orifice 21b. The upright 14 also has a reinforcing rib 22 projecting vertically on the external face 18 side.

The tongue 15 is substantially flat and rectangular. It has an upper face 23 and a lower face 24. Four studs 25 protrude upwards from the upper face 23, said studs being located close to the four corners of the tongue 15. The posterior edge 26 of the tongue 15 has a recessed zone 27, arranged in a substantially centred manner between two studs 25. In addition, a depression 28 that has the general shape of a half-disc is formed in the tongue 15, from the upper face 23, facing the recessed zone 27.

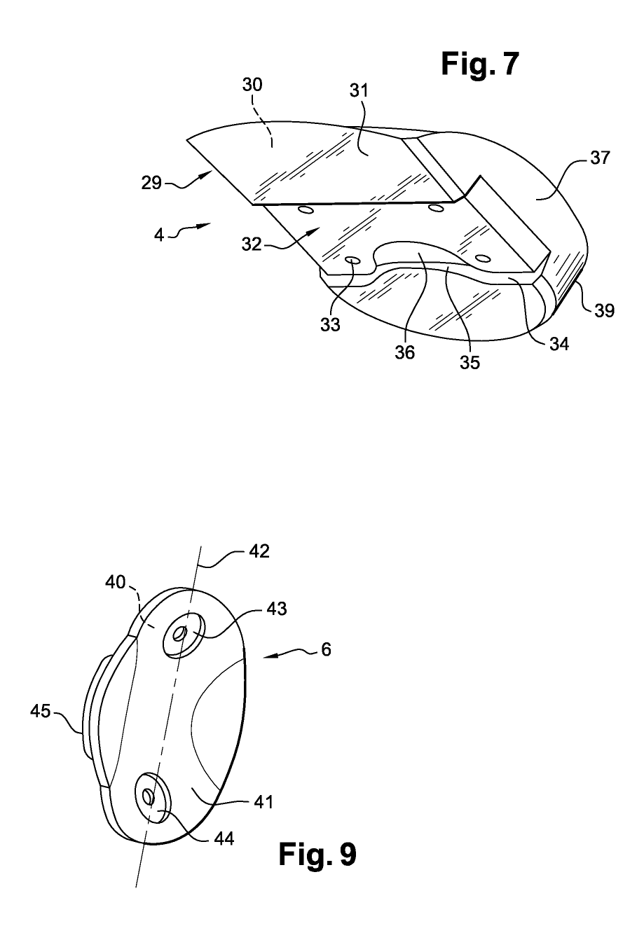

The heel cushion 4, more particularly shown in FIG. 7, is intended to be assembled to the support 3 and to be positioned under the foot. In the embodiment described here, the heel cushion 4 is made from an elastomeric material, which makes it possible to obtain a damping effect and to improve the wearing comfort of the splint 1. It should be noted that the material forming the heel cushion 4 may also have anti-slip properties. This material is for example a silicone or SEBS (styrene ethylene butylene styrene).

The heel cushion 4 comprises a sole 29 having an upper face 30 adapted to the morphology of the foot and a substantially flat lower face 31. A cavity 32 extending transversely from one side of the sole 29 to the other is formed in the heel cushion 4, from the lower face 31 of the sole 29. This cavity 32, which is therefore open in the downward direction, is provided for receiving the tongue 15 of the support 3 and therefore has a shape complementary to this tongue 15. The cavity 32 thus comprises four holes 33, into which the studs 25 can engage, a posterior edge 34 having a zone in relief 35 towards the front arranged in a substantially centred manner between two holes 33, and a boss 36 substantially in the shape of a half-disc extending forwards and downwards from said zone in relief 35.

The tongue 15 is positioned in the cavity 32 and is held in position by the engagement of the studs 25 in the holes 33 and by the cooperation between the zone in relief 35 and the recessed zone 27 on the one hand, and the boss 36 and the depression 28 on the other hand. The support 3 is thus perfectly fixed to the heel cushion 4. In addition, in the mounted position, the lower face 24 of the tongue 15 and the lower face 31 of the sole 29 are located substantially in the same plane, so that the foot of the user rests on a flat surface without any raised areas.

The heel cushion 4 also comprises an internal lateral flange 37, an external lateral flange 38 and a posterior flange 39, which form a housing that receives the foot and holds it in position.

The connecting piece 6, more particularly shown in FIG. 9, is a part made from rigid material, for example made from polyamide. It has a cup shape having a convex external face 40 and a concave internal face 41 and has an axis of symmetry 42. The connecting piece 6 is provided with two through-orifices located on this axis of symmetry 42, namely an upper orifice 43 and a lower orifice 44. Finally, in the embodiment shown, the connecting piece 6 comprises means 45 for attaching a strap 46, said means being arranged on each side of the axis of symmetry 42 and consisting for example of members that protrude outwards and that are provided with a through-slot for the strap.

The connecting piece 6 is mounted between the shell 2 and the support 3, on the internal side, and is assembled to these two parts by means of elements such as rivets. A first rivet is thus engaged in the orifice 12 of the shell 2 and in the upper orifice 43 of the connecting piece 6, said orifices being made to coincide with one another. A second rivet is furthermore engaged in the orifice 20 of the support 3 and in the lower orifice 44 of the connecting piece 6, said orifices being made to coincide with one another.

In this way, the connecting piece 6 is mounted such that it can pivot on the one hand with respect to the shell 2 about a first transverse axis of articulation 47, corresponding to the common axis of the orifices 12, 43, and on the other hand with respect to the support 3 about a second transverse axis of articulation 48, corresponding to the common axis of the orifices 20, 44.

As can be seen in FIG. 2, in the neutral position of use, the axis of symmetry 8 of the shell 2 and the axis of symmetry 16 of the upright 14 of the support 3 extend in substantially vertical directions which are offset from one another in the direction D2, the axis of symmetry 8 of the shell 2 being located further rearwards. Thus, the axis of symmetry 42 of the connecting piece 6 is inclined with respect to the vertical, rearwards and from the bottom upwards. The angle of inclination must be adapted to the morphology of the foot. It is for example around 10° to 20°. In addition, the first and second axes of articulation 47, 48 are offset from one another vertically and in the anteroposterior direction (in the neutral position of use), the second axis of articulation 48 being located further forwards and further downwards.

The external bearing member 5, more particularly shown in FIG. 8, is attached to the shell 2 and to the support 3, opposite the internal face 41 of the connecting piece 6, and is intended to be positioned against the lateral malleolus.

The external bearing member 5 has a cup shape with a convex external face 49 and a concave internal face 50, substantially mating with the shape of the lateral malleolus. It moreover has an axis of symmetry 51. The external bearing member 5 is provided with means for assembly to the shell 2 and to the support 3, which means may be in any suitable form. In the embodiment shown, these assembly means consist of transverse lugs 52 which are directed towards the external side and are equipped with a locking collar 53 at their free end. These lugs 52 are distributed in a set of two upper lugs, engaging in the orifices 13a, 13b of the shell, and a set of two lower lugs, engaging in the orifices 21a, 21b of the upright 14 of the support 3.

The external bearing member 5 is made from a semi-rigid material having elastomeric properties, for example silicone or SEBS.

The external bearing member 5 has a dual function. On the one hand, this bearing member 5 makes it possible to keep the shell 2 and the support 3 in the neutral position of use when the ankle is in the neutral position. On the other hand, this bearing member 5 is designed to deform elastically when the ankle is in flexion or in extension, so as to allow the pivoting of the connecting piece 6 about the first and second axes of articulation 47, 48, and to return the splint 1 to its neutral position when the ankle returns to the neutral position.

It will thus be understood that the rigidity of the external bearing member 5 must be sufficient to carry out the functions of holding and returning to the neutral position. However, the external bearing member 5 must also be sufficiently flexible to be able to deform and thus to allow the double pivoting of the splint 1. In addition, the flexibility of the external bearing member 5 makes it possible to improve the wearing comfort of the splint 1 due to the damping effect provided, and due to the more pleasant contact against the lateral malleolus.

Finally, the internal shell 7 will be described, which is shown more particularly in FIGS. 1 and 3. This is a part made from rigid material, for example made from polyamide, which is intended to be positioned against the internal face of the leg. The internal shell 7 has substantially the same shape as the external shell 2, but the presence of orifices is not necessary.

The two shells 2, 7 are assembled to one another by at least one strap, in this case a median strap 54 and an upper strap 55. Means for attaching these straps may be provided on one of the shells or on each of the shells 2, 7.

According to one embodiment that is not shown, it is possible to provide in addition an internal bearing member intended to be positioned against the medial malleolus and assembled to the other constituent parts of the splint by a strap (for example the strap 46). This internal bearing member would be made from semi-rigid material, for example from elastomeric material such as a silicone or SEBS, and could advantageously have a cup shape with a convex external face and a concave internal face.

The splint 1 is attached to the leg and the foot of the user by a strap 46 which can surround the zone located close to the ankle, passing through the attachment means 45 formed on the connecting piece 6 and through the attachment means 58 formed on the internal bearing member 7.

This splint 1 makes it possible to liberate the movements of flexion/extension of the ankle, so as to allow walking and more generally a return to a sporting activity. The valgus movement is also permitted since it has no negative effect on the injury to the external lateral ligament of the ankle. This movement is nevertheless limited by the rigidity of the splint 1.

On the contrary, the varus movement is greatly limited since it could give rise to a reoccurrence of the injury. A small degree of varus is nevertheless permitted so that the splint 1 can be worn by users that have a slight natural varus.

It should be noted that the splint 1 according to the invention is not very bulky since it comprises just one shell 2 (on just one side of the leg) and since the sole 39 of the heel cushion 4 extends only under approximately half the foot. The splint 1 can therefore be worn inside a shoe without any problem.

It goes without saying that the invention is not limited to the embodiment described above by way of example but rather on the contrary encompasses all variant embodiments. It would be possible in particular to provide stops that limit the relative displacement of the shell 2 and of the support 3, and/or another strap for attaching the splint.

1. Ankle splint comprising:
a shell intended to be positioned against the external face of the leg;
an L-shaped support comprising an upright intended to be positioned against the external face of the foot and a tongue intended to be positioned under the foot;
a connecting piece connecting the shell and the support, said connecting piece being mounted such that it can pivot with respect to the shell about a first transverse axis of articulation and mounted such that it can pivot with respect to the support about a second transverse axis of articulation;
wherein the first and second axes of articulation are offset from one another vertically and in the anteroposterior direction when the splint is positioned on the ankle of a user, said ankle being in the neutral position.

2. Splint according to claim 1, wherein the second axis of articulation is offset towards the front relative to the first axis of articulation.

3. Splint according to claim 1 wherein, in the neutral position of use, the shell and the upright of the support extend in substantially vertical directions which are offset from one another in the anteroposterior direction.

4. Splint according to claim 1, wherein the splint comprises a bearing member arranged opposite the internal face of the connecting piece and attached to the shell and to the support, said bearing member, made from a semi-rigid material, being designed to keep the shell and the support in the neutral position of use when the ankle is in the neutral position, to be able to be elastically deformed so as to allow the pivoting of the connecting piece about the first and second axes of articulation when the ankle is in flexion or in extension and, from this deformed position, to return the splint to its neutral position when the ankle returns to the neutral position.

5. Splint according to claim 4, wherein the bearing member has a concave internal face which substantially mates with the shape of the lateral malleolus.

6. Splint according to claim 1, wherein the splint further comprises a heel cushion intended to be assembled to the tongue of the support and to be positioned under the foot.

7. Splint according to claim 6, wherein the heel cushion is made from an elastomeric material.

8. Splint according to claim 6, wherein the heel cushion comprises a sole intended to be positioned under the foot, which sole has a transverse cavity that is open in the downward direction in the position of use, said cavity being designed to receive the tongue of the support so that, in the mounted position, the lower face of the tongue of the support is located substantially in the same plane as the lower face of the sole.

9. Splint according to claim 6, wherein the sole and the tongue of the support comprise means for reciprocal assembly by engagement.

10. Splint according to claim 6, wherein the heel cushion comprises an internal lateral flange and/or external lateral flange and/or a posterior flange.

11. Splint according to claim 1, wherein the connecting piece comprises means for attaching a strap.

12. Splint according to claim 1, wherein the splint comprises an additional shell intended to be positioned against the internal face of the leg, substantially opposite the shell, by being assembled to this shell by at least one strap.
